(19) United States
(12) Patent Application Publication
(10) Pub. No.: US 2006/0099617 A1
(43) Pub. Date: May 11, 2006

(54) Method of test for animal-derived ingredients in foods

(75) Inventors: Yang-Chih Shih, Taipei City (TW); Lih-Ching Chiueh, Taipei City (TW); Hsu-Yang Lin, Taipei City (TW); Che-Yang Lin, Taichung County (TW); Tsung-Hsi Wu, Taipei City (TW); Yuan-Hsin Chang, Taipei City (TW)

Correspondence Address: HUSCH & EPPENBERGER, LLC, 190 CARONDELET PLAZA, SUITE 600, ST. LOUIS, MO, 63105-3441, US

(73) Assignee: Bureau of Food and Drug Analysis, Department of Health, Executive Yuan, Taipei City (TW)

(21) Appl. No.: 11/234,787

(22) Filed: Sep. 23, 2005

(30) Foreign Application Priority Data
Sep. 24, 2004 (TW) 093128997

(51) Int. Cl. C12Q 1/68

(52) U.S. Cl. 435/6

(57) ABSTRACT

The present invention relates to a method for testing the presence or absence of an animal-derived ingredient(s) in food, which comprises the step of (i) detecting whether a DNA fragment highly reserved among animals is present, and optionally the step of (ii) detecting whether a DNA fragment highly reserved among mammals and poultry is present, and optionally the step of (iii) detecting whether a DNA fragment highly reserved in a specific animal species is present.

### CROSS-REFERENCE TO RELATED APPLICATIONS

This applications claims the benefit of Taiwan application serial no. 093128997, filed Sep. 24, 2004.

### BACKGROUND OF THE INVENTION

1. Field of the Invention

The present invention relates to a method for testing the presence or absence of an animal-derived ingredient(s) in food, which comprises the step of (i) detecting whether a DNA fragment highly conserved among animals is present, and optionally the step of (ii) detecting whether a DNA fragment highly conserved among mammals and poultry is present, and optionally the step of (iii) detecting whether a DNA fragment highly conserved in a specific animal species is present.

2. Background of the Invention

For the motivations of religion, health, economy or eco-environmental protection, the number of “vegetarian foods” or non-animal-derived foods consumers all over the world is increasing, and thereby the production and sales volume of “vegetarian diets” or non animal-derived foods are growing as well. In recent years, various “vegetarian diets” or non animal-derived foods come out constantly, and tend to be more diversified and refined. For example, the products on the market, such as vegetarian ham, vegetarian meat ball, vegetarian fish steak, vegetarian chicken, vegetarian sausage, vegetarian steak, imitate the appearances and tastes of the meat foods so exactly that there seems no difference. However, is it always true that animal-derived ingredients are not present in these so-called “vegetarian diets” or non animal-derived foods? Particularly, some manufactures may adulterate their products with animal-derived ingredients deliberately or indeliberately in order to make their products more attractive to consumers with better tastes and flavors or to facilitate the manufacture and reduce the cost, which more directly infringe the rights and interests of the consumers of “vegetarian diets” or non animal-derived foods. The consumers often doubt whether the commercially available “vegetarian diets” or non animal-derived foods of all sorts are real and whether they contain animal-derived ingredients or not. Therefore, there is a need to develop a testing technology for quickly testing animal-derived ingredients in foods so as to monitor the commercially available “vegetarian diets” or non animal-derived foods, thus accusing and interdicting the illegal proprietors and ensuring the consumers' rights and interests.

In the past, the issue on the adulteration of “vegetarian diets” or non animal-derived foods was not paid much attention to, and the requirement for testing and verifying these products was not high, so there was no specific method directed to detecting the presence of animal-derived ingredients among “vegetarian diets” or non animal-derived foods, but only methods or study for identifying and testing particular animals, animal-derived ingredients or a certain kind of animal, for example, the method for identifying the species of meat products.

The methods for identifying the species of meat products in the past were carried out mainly by utilizing morphology, protein methods (e.g. One-dimensional Protein Electrophoresis and Antigen-antibody test of Immunoserology), chemical methods (e.g. High Performance Liquid Chromatography (HPLC)), and the like. However, protein denaturation often occurs in animal meat products during processing, which makes the species of meat products unable to be indentified effectively by morphology, protein methods and chemical methods mentioned above.

Recently, because of the advancement of the molecular biotechnology, the above problems can be solved by utilizing a small amount of specimen DNA, i.e. performing effectively the related detecting technology based on DNA. The molecular biotechnology-based methods include, for example, DNA hybridization technique (see, Trends in Food Science & Technology. 11:67-77), sequencing technique of PCR products (e.g., the identification of tuna disclosed by J. Agric. Chem. 50:963-969), PCR-Restriction Fragment Length Polymorphism (PCR-RFLP) (e.g., the identification of pigs, cattle and sheep disclosed by J. Agric. Food Chem. 51:1771-1776, J. AOAC Int. 78:1542-1551, J. Agric. Food Chem. 51:1524-1529, and J. Food Prot. 66:682-685), Species-Specific primer PCR-technique (e.g., the identification of pigs, cattle, sheep, chickens, horses disclosed by J. Food Prot. 66:103-109, J. Agric. Food Chem. 49:2717-2721 and Meat Sci. 51:143-148), PCR-SSCP technique (e.g., the identification of fish, for example, disclosed by Food Chem. 64:263-268), Random Amplified Polymorphic DNA technique (RAPDs) (e.g., the identification of mussel and poultry disclosed by J. Agric. Food Chem. 50:1780-1784 and Poult Sci. 80:522-524), Actin-detecting technique (e.g. the identification of chickens disclosed by Meat Sci. 53:227-231), Real-time PCR technique (e.g. Bundesgesundheitsblatt Gesundheitsforsch. Gesundheitsschutz. pp. 1-25), which discloses the real-time PCR method applying the TaqMan probe system, which uses the myostatin gene as the internal control of the experiment, and states that the PCR carried out with the primer pairs designed by the experiment allows for detecting mammals and poultry, the DNA Chip, etc.

The techniques described above are all performed particularly for specific animal species (e.g. pigs, cattle, sheep, chickens and horses) or a certain animal group (e.g. poultry). By implementing these detecting techniques individually, it can only verify whether particular animal species-derived ingredients are present in the samples to be tested, but cannot identify whether the ingredients derived from any possible animal species are present in the samples to be tested.

In addition, Bottero, M. T. et al. used 16 S ribosomal RNA gene as the testing standard and used PCR method to detect the animal tissues (Feeds. J. Food Prot. 66: 2307-2312). However, this experiment is used to detect the animal tissues in the feed for ruminants, but not to test the animal-derived ingredients in foods.

Thus, it is desirable to develop a method for simply, quickly and practically detecting whether animal-derived ingredients are present in foods.

### SUMMARY OF THE INVENTION

The present invention provides a method for testing the presence or absence of an animal-derived ingredient(s) in food, it includes the step of detecting whether a DNA fragment highly conserved among animals is present.

### BRIEF DESCRIPTION OF THE DRAWING

FIG. 1 is a flow chart of the method for detecting the animal-derived ingredient(s) in food, in which “+” represents the detecting result is positive, and “−” represents that the detecting result is negative. According to the flow chart, if one or two of the results of the screening experiment are positive, an animal-derived ingredient is present in the test sample.

### DETAILED DESCRIPTION OF THE INVENTION

The term “a DNA fragment highly conserved among animals” used herein refers to any DNA fragment derived from a highly conserved gene commonly occuring among animals, e.g. genes in mitochondria of animals, whose DNA sequence is highly conserved in each species in most cases, e.g. 12S ribosomal RNA gene, 16S ribosomal RNA gene, 18S ribosomal RNA gene and cytochrome b gene. The highly conserved genes commonly occuring in animals can be verified by comparing with the DNA sequence database or genbank and used as test targets for the testing method of the present invention. Preferably, the test targets in the method of the invention is the DNA fragment derived from 12S ribosomal RNA gene, 16S ribosomal RNA gene, 18S ribosomal RNA gene or cytochrome b gene. Most preferred is the DNA fragment derived from 16S ribosomal RNA gene.

According to the present invention, by testing with the DNA fragment highly conserved among animals as the targets to be detected, the positive result indicates that an animal-derived ingredient is present in the tested sample, and the negative result indicates that an animal-derived ingredient is absent from the tested sample.

Optionally, the method of the invention may also selectively include the step of detecting whether a DNA fragment highly conserved among mammals and poultry is present, so as to initially screen the animal species possibly contained in the food specimen.

The term “a DNA fragment highly conserved among mammals and poultry” refers to any DNA fragment of a highly conserved gene commonly occurring in mammals and poultry. The highly conserved genes commonly occurring in animals can be verified by comparing with the DNA sequence database or genebank and then used as test targets for the testing method of the present invention. For example, The highly conserved DNA fragment commonly occurring in mammals and poultry can be found by comparing with myostatin gene and then used as a test target for the testing method of the present invention.

According to the invention, the optional step of detecting whether a DNA fragment highly conserved among mammals and poultry is present in food can be carried out simultaneously with the step of detecting whether a DNA fragment highly conserved among animals is present in food. Alternatively, the optional step of detecting whether a DNA fragment highly conserved among mammals and poultry is present in food and the step of detecting whether a DNA fragment highly conserved among animals is present in food can be carried out sequentially. That is, after verifying whether a highly conserved DNA fragment of animals is present in foods, the optional step of detecting whether a DNA fragment highly conserved among mammals and poultry is present in food is then carried out.

According to the present invention, if the detecting result of the DNA fragment highly conserved among animals is positive, and the detecting result of the DNA fragment highly conserved among mammals and poultry is also positive, the ingredient(s) derived from mammals and/or poultry including but not limited to pigs, cattle, sheep, horses, kangaroos, rabbits, deer, murines, and chickens, ducks, geese, turkeys and pigeons, is (are) present. In addition, the test sample may also simultaneously contain the ingredient(s) derived from the amphibian and/or aquatic animals, including but not limited to frogs, fishes, cuttlefishes, shrimps, crabs and the like.

According to the invention, if the detecting result of the DNA fragment highly conserved among animals is positive, while the detecting result of the DNA fragment highly conserved among mammals and poultry is negative, the ingredient(s) derived from mammals and poultry is (are) not present, but the ingredient(s) derived from the amphibian and/or aquatic animals, including but not limited to frogs and fishes, cuttlefishes, shrimps, crabs and the like, is (are) present.

Optionally, the present invention may selectively include the step of detecting whether a DNA fragment highly conserved in a particular animal species is present in food to verify the source of the animal ingredient(s) contained in the food.

The term “a DNA fragment highly conserved in a particular animal species” refers to any DNA fragment derived from highly conserved gene commonly occurring in a particular animal species. The highly conserved gene commonly occurring in a particular animal species can be verified by comparing with the DNA sequence database or genebank and then used as a test target for the testing method of the present invention. For example, the DNA fragment of the highly conserved gene commonly occurring in a particular animal species can be found by comparing with the genes in mitochondria of animals or the myostatin gene, or βactin gene, myosin gene or haemoglobin gene, and the like, and then used as a detection target for the testing method of the present invention. In addition, the known DNA fragment of highly conserved genes in particular animal species used in the techniques for verifying various particular animal species described above can also be employed.

According to the present invention, if the detecting result of the DNA fragment highly conserved among animals is positive, and the detecting result of the DNA fragment highly conserved among mammals and poultry is also positive, the DNA sequence highly conserved in the particular species of mammals and/or poultry is used as the detecting target in order to verify the source of the animal-derived ingredient(s) contained in the sample. Optionally, the DNA sequence highly conserved in the particular species of amphibian and/or aquatic animals can also be taken as the detecting object to perform the verification experiment, wherein mammals include but not limited to pigs, cattle, sheep, horses, kangaroos, rabbits, deers and murines; poultry includes but not limited to chickens, ducks, geese, turkeys and pigeons; amphibian includes but not limited to frogs; and aquatic animals include but not limited to fishes, cuttlefishes, shrimps and crabs.

According to the present invention, if the detecting result of the DNA fragment highly conserved among animals is positive, while the detecting result of the DNA fragment highly conserved among mammals and poultry is negative, the DNA sequence highly conserved in a particular species of amphibian and/or aquatic animals is used as the detecting object to verify the source of the animal-derived ingredient(s) contained in the sample, wherein the amphibian includes but not limited to frogs; and the aquatic animals include but not limited to fishes, cuttlefishes, shrimps and crabs.

Any known methods can be used to extract DNA from the food and any method for detecting the presence of DNA, such as PCR, real-time PCR and DNA sequencing technique, may be applied to the present invention to detect the DNA fragments highly conserved among animals, the DNA fragments highly conserved among mammals and poultry and the DNA fragments highly conserved in a particular animal species.

The invention is now further illustrated with the following examples, which are not intended to limit the scope of the invention.

### EXAMPLE

### Example 1

### Test for the Presence of an Animal-Derived Ingredient(s) in the Vegetarian Diet

1. The Sources of the Vegetarian Diet

From June to September in 2004, each local health bureau made a spot check on the vegetarian products, which included vegetarian ham, vegetarian meat ball, vegetarian fish steak, vegetarian chicken, vegetarian sausage, vegetarian hot dog, vegetarian steak and the like (with 90 pieces in total), from the markets and factories of vegetarian diets.

2. Pretreatment of the Samples

An appropriate amount of particular samples were lyophilized for 36 hours, and then ground into powder, and 25 milligrams of the powder therefrom was weighed by a microbalance as the specimen. The step was repeated twice.

3. Extraction of DNA from the Specimens

DNeasy®Tissue kit and the reagents, materials (ATL agent, protein K reagent, AL reagent, DNeasy spin column), collecting tube, AW1, AW2 and AE agent contained therein were employed.

4. Screening Experiment of an Animal Ingredient(s)

In this embodiment, a 16S ribosomal RNA gene commonly occuring in animals was used as the detecting target, and the particular primers SF and SR for detecting were designed for the highly conserved DNA sequence of the gene as shown in Table 1, and the PCR experiment was performed by taking the specimen DNA described above as the template.

TABLE 1

| pri- | | | Ampli- |
|---|---|---|---|
| mer | Sequence 5′-3′ | specificity | con (bp) |
| SF | AagAcgAgaAgaCccT(a/g)tGga(A/G)ctTta | 16 S ribosomal | |
| | | RNA gene/ | |
| SR | GatTgcGctGttAtcCctAggGta | sense strand | |
| SP | FAM- | 16 S ribosomal | 234-265 |
| | Tt(c/t)GgtTggGgtGacCtcGg(a/g)Gt- | RNA gene/anti- | bp |
| | TAMRA | sense strand | |
| | | 16 S | |
| | | ribosomal RNA | |
| | | gene | |

The formulation of the PCR reaction solution:

| | |
|---|---|
| Template DNA (total amount 100 ng) | 5.0 μL |
| 10 × PCR buffer (containing 15 mM MgCl2) | 2.5 μL |
| Taq DNA polymerase (2U/μL) | 2.0 μL |
| 2.5 mM dNTP | 4.0 μL |
| 5 μM primer F | 1.0 μL |
| 5 μM primer R | 1.0 μL |
| sterile pure water | 9.5 μL |
| total volume | 25.0 μL |

Conditions of the PCR reaction:

| | | |
|---|---|---|
| 95° C. | 5 min | 1 cycle |
| 95° C. | 30 sec | |
| 60° C. | 30 sec | 40 cycles |
| 72° C. | 30 sec | |
| 72° C. | 7 min | 1 cycle |

After completing the PCR reaction, the PCR-amplified products were taken out for gel electrophoresis. The size of the PCR-amplified products was observed and compared with that of the positive reaction control, and when the size of the PCR-amplified products of the specimen DNA is the same as that of the positive reaction control (between 234 and 265 bp), it may be determined primarily that an animal-derived ingredient was present in the tested sample.

In this embodiment, the 16S ribosomal RNA gene commonly occuring in animals was used as the detecting target, and a specific TaqMan probe SP was designed, in addition that the specific primers SF and SR for detecting were designed for the highly conserved DNA sequence of the gene, as shown in table 1. The real-time PCR experiment was performed with the above-mentioned specimen DNA as the template by using a Roche Light Cycler real-time PCR reaction instrument.

The formulation of the real-time PCR reaction solution:

| | |
|---|---|
| Template DNA | 5.0 μl |
| 25 mM MgCl2 solution | 2.4 μL |
| LightCycler-FastStart DNA Master | 2.0 μL |
| Hybridization Probes | |
| 5 μM primer F | 1.5 μL |
| 5 μM primer R | 1.5 μl |
| 3.3 μM probe | 1.5 μL |
| sterile pure water | 6.1 μL |
| total volume | 20.0 μL |

Conditions of the real-time PCR reaction:

| | | |
|---|---|---|
| 95° C. | 10 min | 1 cycle |
| 95° C. | 5 sec | |
| 60° C. | 25 sec | 45 cycles |
| 72° C. | 8 sec | |
| 35° C. | 45 sec | 1 cycle |

After the real-time PCR reaction was completed, the fluorescence amplification curve of the real-time PCR reaction of the specimen DNA was compared with that of the positive reaction control. If the real-time PCR reaction result of the specimen DNA shows a increasing tendency, it may be determined that the animal-derived ingredient(s) is (are) present in the test sample.

5. Screening Experiment of the Ingredient(s) of Mammals and Poultry

In this embodiment, the myostatin gene commonly occuring in mammals and poultry was used as the detecting target. The specific primers MYE and MYR for detecting were designed for the highly conserved DNA sequence of the gene as shown in Table 2 and the PCR experiment was performed by taking the specimen DNA described above as the template.

TABLE 2

| | | | Amplicon |
|---|---|---|---|
| primer | Sequence 5′-3′ | Specificity | (bp) |
| MYF | TtgTgcAaaTccTgaGacTcaT | Myostatin gene/ | |
| | | sense strand | |
| MYR | AtaCcaGtgCctGggTtcAt | Myostatin gene/ | 97 bp |
| | | anti-sense strand | |
| MYP | FAM-CccAtgAaaGacGgtAca | Myostatin gene | |
| | AggTatActG-TAMRA | | |

The formulation of the PCR reaction solution:

| | |
|---|---|
| Template DNA (total amount 100 ng) | 5.0 μL |
| 10 × PCR buffer (comprising 15 mM MgCl2) | 2.5 μL |
| Taq DNA polymerase (2U/μL) | 2.0 μL |
| 2.5 mM dNTP | 4.0 μL |
| 5 μM primer F | 1.0 μL |
| 5 μM primer R | 1.0 μL |
| sterile pure water | 10.5 μL |
| total volume | 25.0 μL |

Conditions of the PCR reaction

| | | |
|---|---|---|
| 95° C. | 5 min | 1 cycle |
| 95° C. | 30 sec | |
| 60° C. | 30 sec | 40 cycles |
| 72° C. | 30 sec | |
| 72° C. | 7 min | 1 cycle |

After completing the PCR reaction, the PCR-amplified products were taken out for gel electrophoresis. The size of the PCR-amplified products was observed and compared with that of the positive reaction control, and when the size of the PCR-amplified products of the specimen DNA was the same as that of the positive reaction control (i.e. 97 bp), it may be determined primarily that the animal-derived ingredient(s) of mammals or poultry is (are) present in the tested sample, and meanwhile the animal-derived ingredient(s) of the amphibian and/or aquatic animals is (are) also probably present in the test sample.

In the embodiment, the myostatin gene commonly occuring in mammals and poultry was used as the detecting target, and a specific primer TaqMan probe MYP was designed. In addition, the specific primers MYF and MYR for detecting were designed aiming at the DNA sequence highly conserved in the gene, as shown in table 2. The above specimen DNA was used as the template and the real-time PCR experiment was performed by using a Roche Light Cycler real-time PCR reaction instrument.

The formulation of the real-time PCR reaction solution:

| | |
|---|---|
| Template DNA | 5.0 μL |
| 25 mM MgCl2 solution | 2.4 μL |
| LightCycler-FastStart DNA Master | 2.0 μL |
| Hybridization Probes | |
| 5 μM primer F | 1.5 μL |
| 5 μM primer R | 1.5 μL |
| 3.3 μM probe | 1.5 μL |
| sterile pure water | 6.1 μL |
| total volume | 20.0 μL |

Conditions of the real-time PCR reaction:

| | | |
|---|---|---|
| 95° C. | 10 min | 1 cycle |
| 95° C. | 5 sec | |
| 54° C. | 25 min | 45 cycles |
| 72° C. | 8 sec | |
| 35° C. | 45 sec | 1 cycle |

After the real-time PCR reaction was completed, the fluorescence amplification curve of the real-time PCR reaction of the specimen DNA was compared with that of the positive reaction control. If the real-time PCR reaction result of the specimen DNA shows a increasing tendency, it can be determined that the animal-derived ingredient(s) of mammals or poultry is (are) present in the tested sample, and meanwhile the animal-derived ingredient(s) of the amphibian and/or aquatic animals is (are) also probably present in the tested sample.

6. Verification Experiment of a Particular Species

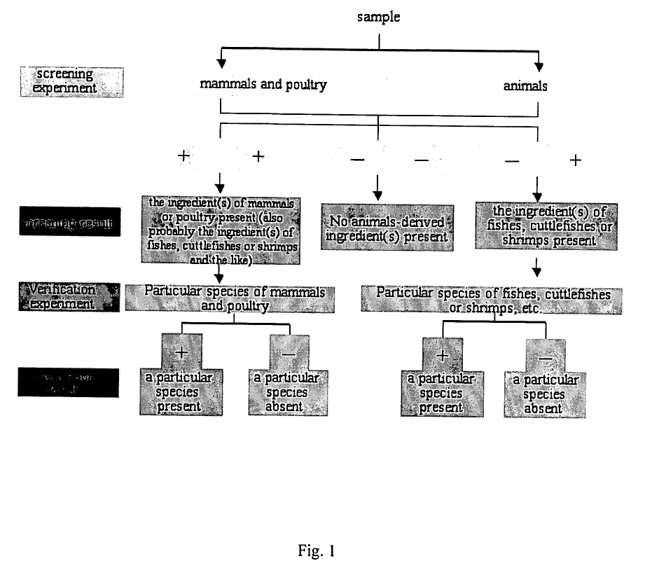

There were three combinations in the above testing results as shown in FIG. 1. First, if the results of the screening experiments of the animal ingredient(s) and the ingredient(s) of mammals and poultry are both negative, it can be determined that no animal-derived ingredient is present in the test sample. Second, if the results of the screening experiments of the animal ingredient(s) and the ingredient(s) of mammals and poultry are both positive, it can be determined that the animal-derived ingredient(s) of mammals or poultry is (are) present in the test sample, and meanwhile the ingredient(s) derived from the amphibians, fishes and/or aquatic animals is (are) also probably present. Third, if the result of the screening experiment of the animal ingredient(s) is positive, while the screening experiment of the ingredient of mammals and poultry is negative, it can be determined that the ingredient(s) derived from the amphibians, fishes and/or aquatic animals is (are) present in the test sample.

In the embodiment, the DNA sequences highly conserved in pigs, chickens and cattle were used as detecting targets, and the real-time PCR method was used to perform the verification experiment. When the real-time PCR reaction was completed, the fluorescence amplification curve of the real-time PCR reaction of the specimen DNA was compared with that of the positive reaction control. If the real-time PCR reaction result of the specimen DNA shows a increasing tendency, it can be determined that the ingredient(s) of pigs, chickens or cattle is (are) present in the test sample.

In addition, in this embodiment, the PCR-amplified products from the above screening experiments of the animal ingredient(s) were recovered and purified prior to DNA sequencing, and the obtained DNA sequences were input into the database for species alignment. If the comparison results match the DNA sequences of pigs, chickens, cattle or fishes, it can determined respectively that the ingredient(s) derived from pigs, chickens, cattle or fishes is (are) present in the test sample.

7. Results

According to the test for the presence of animal-derived ingredient(s) in vegetarian food implemented according to the present invention, 90 types of vegetarian foods on the market were tested in total during the period of June to September in 2004, wherein 37 of them contained the animal-derived ingredients, in which 12 contained pig-derived ingredients, 6 contained chicken-derived ingredients, 16 contained cattle-derived ingredients and 7 contained fish-derived ingredients, as shown in table 3.

TABLE 3

| | Location of | Testing | Quantity | Testing result (number) | | | | | |
|---|---|---|---|---|---|---|---|---|---|
| No. | Obtaining Samples | Date | of Sample | Negative | Positive | Pig | Cattle | Chicken | Fish |
| 1 | Kaohsiung County | 04.06.16. | 7 | 0 | 7 ^{a} | 6 | 0 | 0 | 2 |
| 2 | Kaohsiung County | 04.06.17. | 11 | 11 | 0 | 0 | 0 | 0 | 0 |
| 3 | Yunlin County | 04.06.17. | 9 | 9 | 0 | 0 | 0 | 0 | 0 |
| 4 | Tainan County | 04.06.18. | 6 | 3 | 3 | 0 | 2 | 1 | 0 |
| 5 | Taoyuan County | 04.06.29. | 10 | 6 | 4 | 0 | 3 | 0 | 1 |
| 6 | Taichung City | 04.07.05. | 1 | 0 | 1 ^{b} | 1 | 0 | 1 | 0 |
| 7 | Taipei City | 04.07.06. | 3 | 1 | 2 | 0 | 2 | 0 | 0 |
| 8 | Taipei City | 04.07.06. | 3 | 2 | 1 | 0 | 1 | 0 | 0 |
| 9 | Taipei County | 04.07.06. | 10 | 3 | 7 ^{c} | 4 | 2 | 1 | 2 |
| 10 | Taichung County | 04.07.07. | 6 | 3 | 3 | 0 | 3 | 0 | 0 |
| 11 | Kaohsiung City | 04.07.08. | 18 | 13 | 5 | 1 | 2 | 2 | 0 |
| 12 | Taoyuan County | 04.07.13. | 3 | 1 | 2 | 0 | 0 | 1 | 1 |
| 13 | Taipei County | 04.07.26. | 2 | 0 | 2 | 0 | 1 | 0 | 1 |
| 14 | Taichung County | 04.09.03. | 1 | 1 | 0 | 0 | 0 | 0 | 0 |

| | | | | | | | |
|---|---|---|---|---|---|---|---|
| Total | 90 | 53 | 37 | 12 | 16 | 6 | 7 |
| | | (59%) | (41%) | (13%) | (18%) | (7%) | (8%) |

Note:
^{a} one sample was detected and contains both pig- and fish-derived ingredients.
^{b} one sample was detected and contains both pig- and chicken-derived ingredients.
^{c} two samples were detected and contain both pig- and fish-derived ingredients.
“Positive”: representing that animal-derived ingredients are present in the sample.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

We claim:

1. A method for testing the presence or absence of an animal-derived ingredient(s) in food, comprising the step of (i) detecting whether a DNA fragment highly conserved among animals is present.

2. The method according to claim 1, further comprising the step of (ii) detecting whether a DNA fragment highly conserved among mammals and poultry is present in the food.

3. The method according to claim 2, further comprising the step of (iii) detecting whether a DNA fragment highly conserved in a particular animal species is present in the food.

4. The method according to claim 1, 2 or 3, wherein the DNA fragment highly conserved among animals is derived from 12S ribosomal RNA gene, 16S ribosomal RNA gene, 18S ribosomal RNA gene or cytochrome b gene.

5. The method according to claim 4, wherein the DNA fragment highly conserved among animals is derived from 16S ribosomal RNA gene.

6. The method according to claim 1, 2 or 3, wherein the DNA fragment highly conserved among mammals and poultry is derived from myostatin gene.

7. The method according to claim 2, wherein the step of (i) detecting whether a DNA fragment highly conserved among animals is present in the food and the step of (ii) detecting whether a DNA fragment highly conserved among mammals and poultry is present are carried out sequentially or simultaneously.

8. The method according to claim 3, wherein when the result of the step of (ii) detecting whether a DNA fragment highly conserved among mammals and poultry is present in the food is positive, the step of (iii) detecting whether a DNA fragment highly conserved in a particular animal species is present in the food is used to detect that whether a DNA fragment highly conserved in a particular species of mammals and/or poultry is present in the food.

9. The method according to claim 8, wherein the particular species of mammals and/or poultry is selected from the group consisting of pigs, cattle, sheep, horses, kangaroos, rabbits, deers, chickens, ducks, geese, turkeys and pigeons.

10. The method according to claim 7, wherein whether a DNA fragment highly conserved in a particular species of amphibian and/or aquatic animals is present in the food is also detected.

11. The method according to claim 10, wherein the particular species of amphibian and/or aquatic animals is selected from the group consisting of frogs, fishes, cuttlefishes, shrimps and crabs.

12. The method according to claim 3, wherein when the result of the step of (ii) detecting whether a DNA fragment highly conserved among mammals and poultry is present in the food is negative, the step of (iii) detecting whether a DNA fragment highly conserved in a particular animal species is present in the food is used to detect whether a DNA fragment highly conserved in a particular species of amphibian and/or aquatic animals is present in the food.

13. The method according to claim 12, wherein the particular species of amphibian and/or aquatic animals is selected from the group consisting of frogs, fishes, cuttlefishes, shrimps and crabs.


SEQUENCE LISTING (6 sequences)

SEQ ID NO: 1 - DNA, 27 bp, Artificial sequence
  PCR primer ′SF′ for highly conserved animal 16 S ribosomal RNA gene sequence/ sense strand
aagacgagaagaccctrtggarcttta

SEQ ID NO: 2 - DNA, 24 bp, Artificial sequence
  PCR primer ′SR“ for highly conserved animal 16 S ribosomal RNA gene sequence/anti-sense strand
gattgcgctgttatccctagggta

SEQ ID NO: 3 - DNA, 23 bp, Artificial sequence
  PCR primer ′SP′ for highly conserved animal 16 S ribosomal RNA gene sequence
ttyggttggggtgacctcggrgt

SEQ ID NO: 4 - DNA, 22 bp, Artificial sequence
  PCR primer ′MYF′ for highly conserved mammalian and poultry Myostatin gene/sense strand sequence
ttgtgcaaatcctgagactcat

SEQ ID NO: 5 - DNA, 20 bp, Artificial sequence
  PCR primer ′MYR′ for highly conserved mammalian and poultry Myostatin gene/anti-sense strand sequence
ataccagtgcctgggttcat

SEQ ID NO: 6 - DNA, 28 bp, Artificial sequence
  PCR primer ′MYP′ for highly conserved mammalian and poultry Myostatin gene sequence
cccatgaaagacggtacaaggtatactg